(19) United States
(12) Patent Application Publication
(10) Pub. No.: US 2014/0066475 A1
(43) Pub. Date: Mar. 6, 2014

(54) Method For Treating Pruritus

(71) Applicant: Euro-Celtique S.A., Luxembourg (LU)

(72) Inventors: Alexander Oksche, Limburg-Linter (DE); Karin Wiedel, Hofheim am Taunus (DE)

(73) Assignee: Euro-Celtique S.A., Luxembourg (LU)

(21) Appl. No.: 14/016,796

(22) Filed: Sep. 3, 2013

Related U.S. Application Data
(60) Provisional application No. 61/696,331, filed on Sep. 4, 2012.

(51) Int. Cl. A61K 31/439; A61K 45/06

(52) U.S. Cl. CPC A61K 31/439; A61K 45/06
USPC 514/295

(57) ABSTRACT

Benzomorphan compounds are found to be useful for treating, ameliorating or preventing pruritus, and in particular pruritus associated with (including induced by) the administration of opioids. Antipruritic activity is believed to be mediated through the dual action of the compounds as mu opioid receptor antagonists and kappa opioid receptor agonists. Pharmaceutical compositions contain therapeutically effective amounts of these useful compounds, optionally in combination with second therapeutic agents, such as opioid or non-opioid analgesics or other compounds.

This application claims the benefit of U.S. provisional Ser. No. 61/696,331 filed 4 Sep. 2012, which is incorporated herein.

### BACKGROUND

This invention relates in general to methods of treating, preventing or ameliorating pruritus (itching) and its consequent scratching. Itch may be caused by a wide variety of dermatological and/or neurological conditions as described herein. While the methods of the invention may be useful in itch of any etiology, they are well-suited for chronic pruritus and, in particular, for pruritus that is a frequently reported side effect of opioid therapy.

Pain is the most common symptom for which patients seek medical advice and treatment. While acute pain is usually self-limited, chronic pain can persist for 3 months or longer and lead to significant changes in a patient's personality, lifestyle, functional ability and overall quality of life (K. M. Foley, Pain, in Cecil Textbook of Medicine 100-107, J. C. Bennett and F. Plum eds., 20th ed. 1996). Pain has traditionally been managed by administering either a non-opioid analgesic (such as acetylsalicyclic acid, choline magnesium trisalicylate, acetaminophen, ibuprofen, fenoprofen, diflunisal or naproxen), or an opioid analgesic (such as morphine, hydromorphone, hydrocodone, methadone, levorphanol, fentanyl, oxycodone or oxymorphone). Various compounds have been found to react with at least three opioid receptors in the body: mu (μ) opioid receptors (also MOR), kappa (κ) opioid receptors (also KOR), and delta (δ) opioid receptors (also DOR). However, use of opioid analgesics can often lead to side effects, such as constipation, urinary retention, dysphoria, and pruritus, among others.

Oral and topical treatments for pruritis are known. Some oral treatments include antihistaminic agents, antiallergic agents, and corticosteroids. Some topical treatments include antihistamines, adrenocortic steroidal medicines, nonsteroidal antiphlogistics, camphor, menthol, phenol, salicylic acid, rectified tar oil, crotamiton, capsaicin, and moisture-retentive agents (e.g., urea, Hirudoid (trade name; a heparinoid from animal organs, available from Maruho Co., Ltd.), and Vaseline).

Opioid administration has been associated with inducement of itching. It is generally thought that opioid agonists initiate itching, while opioid antagonists have an antipruritic activity. But as discussed herein, the specific opioid receptors involved, the involvement of nociceptive receptors and/or pruriticeptive receptors, and the chronic vs. acute etiology of various itch conditions complicate the situation, and leave the sensation of itch poorly understood. Some authors have suggested that pruritus associated with opioid therapy occurs as a result of the action of MOR agonists directly upon mu opioid receptors located in the central nervous system (CNS) (Ko, et al. (2004), The Role of Central μ Opioid Receptors in Opioid-Induced Itch in Primates, Journal of Pharmacology and Experimental Therapeutics, 310:1 pp 169-176).

In addition, U.S. Pat. No. 5,972,954 to Foss, et al. describes certain quaternary opioids, e.g. methylnaltrexone and other quaternary noroxymorphones, as useful for treating opioid-like side effects such as dysphoria, urinary retention, constipation and pruritus. U.S. Pat. No. 6,984,493 to Kumagai, et al. describes the management of opioid-involved itching condition by (1) administration of an antagonist against the mu opioid receptor, (2) inhibition of the synthesis of mu opioid agonist peptides, or (3) administration of a kappa opioid receptor agonist. Of these three, only the administration of the kappa agonist, 17-cyclopropylmethyl-3,14β-dihydroxy-4,5α-epoxy-6β-[N-methyl-trans-3-(3-furyl)acrylamide] morphinan hydrochloride, is exemplified. Kumagai, et al. also describes measuring the ratios of various opioid peptides in the bloodstream as a means to diagnose or confirm a diagnosis of pruritus.

WO Patent publication 2009/023567 and US patent publication 2009/0197905, both claiming priority to application Ser. No. 60/954,960 filed 9 Aug. 2007 and assigned to Rensselaer Polytechnic Institute, each describe certain quaternary opioid carboxamides as useful for ameliorating the side effects of therapeutic opiates, including constipation, emesis, cough suppression, pruritus, dysphoria and urinary retention. Some of the disclosed compounds are benzomorphans and at least one is said to have a relatively high affinity for the mu opioid receptor and a relatively low affinity for the delta opioid receptor.

### SUMMARY OF THE INVENTION

This invention relates to the use of certain compounds and compositions as defined below in the treatment, amelioration or prevention of pruritus of any etiology; and, in particular, pruritus associated with (including induced by) the administration of opioids or other mu agonists.

The present invention also provides the use of such compounds and compositions in the manufacture of a medicament for treating, ameliorating or preventing pruritus, particularly pruritus induced by or associated with the administration of opioids, which pruritus is believed to be mediated via mu opioid receptor agonist activity. Thus, in one embodiment, the invention utilizes the mu receptor antagonist activity of compounds useful in practicing the invention to alleviate the symptoms of pruritus. In another embodiment, such compounds have dual activity as both a mu receptor antagonist and a kappa receptor agonist.

In another embodiment, the present invention provides methods comprising co-administering to a patient both an effective amount of a compound useful in practicing the invention that is a mu antagonist and/or kappa agonist in combination with an analgesically effective amount of a mu agonist. In another embodiment, the method comprises co-administration to a patient of both an effective amount of a compound useful in practicing the invention that is both a mu antagonist and a kappa agonist, and an analgesically effective amount of a mu agonist.

The present invention further provides a method of modulating activity of at least one type of opioid receptor so as to treat, ameliorate or prevent pruritus, comprising exposing the receptor to an effective amount of a compound useful in practicing the invention. In one embodiment, the opioid receptor is a mu receptor. In another embodiment, the receptor is a kappa receptor. In another embodiment, the compound modulates both a mu receptor and a kappa receptor. In another embodiment, the compound antagonizes the mu receptor. In another embodiment, the compound agonizes the kappa receptor. In another embodiment the compound both antagonizes the mu receptor and agonizes the kappa receptor.

The present invention further provides pharmaceutical compositions useful for treating, ameliorating or preventing pruritus, particularly pruritus associated with (including induced by) the administration of opioids or other mu agonists. Such a pharmaceutical composition may comprise an effective amount of a benzomorphan compound useful in practicing the invention admixed with one or more pharmaceutically acceptable carriers or excipients. In one embodiment, the pharmaceutical composition may be a formulation for topical application as described herein.

Various aspects of this invention will become apparent to those skilled in the art from the following detailed description.

### DETAILED DESCRIPTION

Unless defined otherwise, all technical and scientific terms used herein have the same meaning as commonly understood by one of ordinary skill in the art to which the invention belongs. Although any methods and materials equivalent to those described herein may be used in the practice or testing of the present invention, exemplary and illustrative methods and materials are described herein. All references cited herein, including books, journal articles, published U.S. or foreign patent applications, issued U.S. or foreign patents, and any other references, are each incorporated by reference in their entireties, including all data, tables, figures, and text presented in the cited references.

All numerical ranges are understood to include all possible incremental sub-ranges within the outer boundaries of the range. Thus, a range of 30 to 90 mg also describes, for example, 35 to 50 mg, 45 to 85 mg, and 40 to 80 mg, etc.

Pruritus

Pruritus is a condition associated with discomfort and itching of the skin, sometimes manifesting as a severe and intractable itch. The linkage between pain and itch has been well-established in the literature due to the similarities in receptors and spinal pathways. The so-called “intensity theory”—now thought to be untenable —proposed that the same sensors were involved and the distinction between pain and itch was merely one of intensity of the stimulus. This theory has mainly been supplanted by the “selectivity” theory.

The selectivity theory proposes that pruritoceptors are a specialized subset of nociceptors and account for the sensation of itch only. When a stimulus—typically a mechanical, thermal or chemical stimulus—activates these receptors, the sensation of itch is perceived unless the stimulus also activates the larger population of nociceptive receptors, in which case the sensation of pain is perceived. Some authors have proposed that these pruritoceptors and nociceptors are located in topographically different layers of the skin.

Pruritus may also appear in acute and chronic varieties. There is some evidence that acute pruritus is not mediated via opioid receptors the way chronic pruritus seems to be. Chronic pruritus is of greater concern due to the potential for loss of skin integrity from excessive scratching. Many nociceptive pruritic conditions also involve chronic dermatological conditions, including inflammatory dermatitis, contact dermatitis, skin cancers, and others. Some specific dermatological pruritic conditions include: atopic dermatitis, asteatotic dermatitis, ectopic dermatitis, neurodermatitis, seborrheic dermatitis, autosensitization dermatitis, caterpillar dermatitis, senile pruritus, insect bite, poison plant-induced, aquagenic, hydroxyethyl-starch induced, hyperesthesia optica, urticaria, prurigo (e.g. simplex or nodularis), herpes, impetigo, eczema, tinea, lichen, psoriasis (e.g. vulgaris or inverse), xerosis, cutis, macular amyloidosis, scabies, acne vulgaris, and other dermatoses. Also related to contact pruritus is post-burn itch. Cancers such as cutaneous lymphomas, melanomas, or any malignant tumor of the skin or integument can also produce a pruritic condition.

Neuropathic pruritus is itch caused by or associated with disease or failure of certain organs, notably the liver, pancreas and kidneys. Specific pruritic conditions can be triggered by cholestasis, diabetes, nephrogenic or renal failure or uremia. Patients on hemodialysis or peritoneal dialysis often exhibit pruritic symptoms. Another condition associated with itch is pruritus associated with pregnancy.

Systemic causes of pruritus can also include drug-induced itch. Administration of many drugs has been associated with the side effect of pruritus or itching. Allergic reactions may occur against any drug, but particularly against antibiotics like sulfonamides, penicillins, ampicillins, tetracyclines, and neomycin. Drug allergies may cause itch by a histamine-mediated mechanism. Apart from allergies, other drugs are associated with the side effect of pruritus. Notable among these are the anti-malarial drug chloroquine, and the opioids and opiate-like drugs discussed herein. Others include allopurinol, simvastatin, hormones like estrogens, progestins and testosterone, and certain cancer chemotherapies.

Finally, pruritus of unknown or psychic origin is also sometimes referred to as idiopathic pruritus, intractable pruritus, or generalized pruritus.

The methods of the present invention may be used to treat, ameliorate or prevent one or more of the pruritic conditions listed above.

Compounds Useful in Practicing the Invention

The present invention is related to the use of compounds and compositions to treat, ameliorate or prevent conditions of pruritus, including but not limited to pruritus associated with or induced by opioid therapy. Such compounds (herein “compounds useful in practicing the invention”) will generally have a particular profile of interaction with opioid receptors: namely they will bind with mu opioid receptors and cause antagonism (relative to other mu opioid receptor agonists, including exogenous or endogenous peptides such as α- or β-endorphin, enkephalins, endomorphins, etc.); or they will bind with kappa opioid receptors and cause activity or agonism; or, in some embodiments, compounds useful in practicing the invention will be both mu antagonists and kappa agonists.

As used herein, “agonism” is caused by an “agonist” compound, when the compound binds to receptors of the body and mimics the regulatory activity or effects of endogenous ligands on those receptors. In contrast, “antagonism” is caused by an “antagonist” compound, when that compound binds to receptors of the body and, instead of producing the regulatory effect they block the binding of effective ligands to the receptor, thereby decreasing the activity or regulatory effects at the receptor. (Ross and Kenakin, “Ch. 2: Pharmacodynamics: Mechanisms of Drug Action and the Relationship Between Drug Concentration and Effect”, pp. 31-32, in Goodman & Gilman's the Pharmacological Basis of Therapeutics, 10th Ed. (J. G. Hardman, L. E. Limbird and A. Goodman-Gilman eds., 2001). The extent to which a compound binds to a receptor is known as its affinity for the receptor, which is measured by the inhibitor constant, Ki (nM). A lower Ki value indicates higher affinity. The extent to which a compound produces or blocks the production of a regulatory effect at the receptor (i.e. the degree to which it agonizes, partially agonizes or antagonizes the receptor is measure by Emax and EC₅₀. A relatively high Emax—e.g. greater than about 30%—is considered an activator or agonist; whereas a low Emax—e.g. less than about 20%—is considered an antagonist. A partial agonist may have an intermediate Emax.

Compounds useful in practicing the invention may be benzomorphans, such as the quaternized benzomorphans disclosed in U.S. patent application Ser. No. 12/745,472, published as US Patent Application Publication 2010/0324080, the disclosure of which is incorporated herein in its entirety, but also briefly summarized below. Some compounds useful in practicing the invention are benzomorphans defined according to Formula I or a solvate or prodrug thereof,

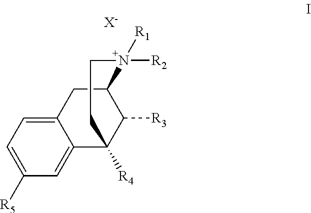

wherein R¹ and R² are each independently selected from the group consisting of —(C₁-C₁₀)alkyl, —(C₂-C₁₀)alkenyl, —(C₂-C₁₀)alkynyl, —(C₃-C₁₂)cycloalkyl, —(C₃-C₁₂)cycloalkenyl, —(CH₂)ₙ—O—(CH₂)ₙ—CH₃, (C₁-C₁₀)alkoxy, C(halo)₃, CH(halo)₂, CH₂(halo), C(O)R⁶, —C(O)O—(C₁-C₁₀)alkyl, and —(CH₂)ₙ—N(R⁷)₂, each of which is optionally substituted by 1, 2, or 3 independently selected R⁸ groups;

R³ and R⁴ are each independently selected from (a)—H; or (b)—(C₁-C₅)alkyl, —(C₂-C₅)alkenyl, and —(C₂-C₅)alkynyl;

R⁵ is selected from (a)—H, —OH, halo, —C(halo)₃, —CH(halo)₂, and—CH₂(halo) (b)—(C₁-C₅)alkyl, —(C₂-C₅)alkenyl, —(C₂-C₅)alkynyl, —(CH₂)ₙ—O—(CH₂)ₙ—CH₃, —(C₁-C₅)alkoxy, each of which is optionally substituted with 1, 2, or 3 independently selected R⁸ groups;

R⁶ is selected from —H, —(C₁-C₁₀)alkyl, —(C₂-C₁₀)alkenyl, —(C₂-C₁₀)alkynyl, and —(C₁-C₁₀)alkoxy;

each R⁷ is independently selected from —H, —(C₁-C₁₀)alkyl, —(C₂-C₁₀)alkenyl, and —(C₂-C₁₀)alkynyl;

each R⁸ is independently selected from —OH, halo, —(C₁-C₁₀)alkyl, —(C₂-C₁₀)alkenyl, —(C₂-C₁₀)alkynyl, —(C₁-C₁₀)alkoxy, —(C₃-C₁₂)cycloalkyl, —CHO, —C(O)OH, —C(halo)₃, —CH(halo)₂, CH₂(halo), and —(CH₂)ₙ—O—(CH₂)ₙ—CH₃;

X⁻ is a pharmaceutically acceptable anion; and

each n is independently selected from an integer from 0, 1, 2, 3, 4, 5, or 6;

provided that the compound is a mu opioid receptor antagonist and a kappa opioid receptor agonist and the compound is not

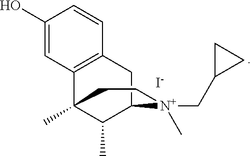

In one embodiment, at least one of R¹ and R² is a (C₁-C₁₀)alkyl substituted with at least one R⁸ group. In another embodiment R⁸ is selected as —(C₃-C₁₂)cycloalkyl. In particular embodiments, R⁸ is selected from cyclopropyl, cyclopentyl, cyclohexyl, cycloheptyl, cyclooctyl, cyclononyl and cyclodecyl, provided that when R³ and R⁴ are each —CH₃ and R⁵ is OH, then R¹ is not —CH₃ when R² is methyl substituted with cyclopropyl.

In another embodiment, at least one of R¹ and R² is a —(C₂-C₁₀)alkenyl. In another embodiment, at least one of R¹ and R² is a —(C₂-C₅)alkenyl. In another embodiment at least one of R¹ and R² is —CH₂-cyclopropyl, —CH₂CH₂-cyclopropyl, and CH₂CH₂CH₂-cyclopropyl, provided that when one of R¹ and R² is —CH₂-cyclopropyl and R³ and R⁴ are each —CH₃ and R⁵ is OH, then the other of R¹ or R² is not CH₃.

In another embodiment, R³ and R⁴ are each independently selected from a —(C₁-C₅)alkyl. In an alternative embodiment, each of R³ and R⁴ is independently selected from methyl, ethyl, and propyl.

In another embodiment, R⁵ is —OH.

In another embodiment, R⁵ is —(CH₂)ₙ—O—(CH₂)ₙ—CH₃. In an alternative embodiment, R⁵ is selected from —(CH₂)—O—CH₃ and —(CH₂)—O—(CH₂)—CH₃.

In one embodiment wherein R¹, R³ and R⁴ are each —CH₃ and R⁵ is —OH, R² is not —CH₂—CH═C(CH₃)₂.

In another embodiment wherein R², R³ and R⁴ are each —CH₃ and R⁵ is —OH, R¹ is not —CH₂—CH═C(CH₃)₂.

In another embodiment wherein R¹ is selected from —CH₃ or —CD₃, R³ and R⁴ are each selected as —CH₃, and R⁵ is —OH, R² is not —CH₃ or —CD₃;

In another embodiment wherein R¹ is selected as —CH₃ or —C₂H₅, R³ and R⁴ are each selected as —CH₃, and R⁵ is —OH, R² is not —CH₃ or —C₂H₅; and

In another embodiment wherein R¹, R², R³, and R⁴ are each selected as —CH₃, then R⁵ is not -halo.

In another embodiment, each n is independently selected from 1, 2 and 3.

In another embodiment, X⁻ is a pharmaceutically acceptable anion selected from organic and inorganic anions, such as sulfate; citrate; acetate; dichloroacetate; trifluoroacetate; oxalate; halide, such as chloride, bromide, iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; gluconate; glucoronate; saccharate; formate; mandelate; arginate; carboxylate; benzoate; glutamate; methanesulfonate; ethanesulfonate; benzenesulfonate; p-toluenesulfonate; and pamoate (i.e., 1,1′-methylene-bis-(2-hydroxy-3-naphthoate)).

A specific compound useful in practicing the invention is: 3-allyl-9-hydroxy-3,6,11-trimethyl-1,2,3,4,5,6-hexahydro-2,6-methano-benzo[d]azocinium; and the pharmaceutically acceptable salts, solvates and prodrugs thereof.

As used herein, the term “(C₁-C₁₀) alkyl” refers to straight-chain and branched non-cyclic saturated hydrocarbons having from 1 to 10 carbon atoms. Representative straight chain —(C₁-C₁₀) alkyl groups include methyl, -ethyl, -n-propyl, -n-butyl, -n-pentyl, -n-hexyl, n-heptyl, n-octyl, n-nonyl and n-decyl. Representative branched —(C₁-C₁0)alkyl groups include isopropyl, sec-butyl, isobutyl, tert-butyl, isopentyl, neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, and 3,3-dimethylbutyl.

As used herein, the term “(C₁-C₅)alkyl” refers to straight-chain and branched non-cyclic saturated hydrocarbons having from 1 to 5 carbon atoms. Representative straight chain —(C₁-C₅)alkyl groups include methyl, -ethyl, -n-propyl, -n-butyl, and -n-pentyl. Representative branched-chain —(C₁-C₅)alkyl groups include isopropyl, sec-butyl, isobutyl, tert-butyl, isopentyl, neopentyl, 1-methylbutyl, 2-methylbutyl, 3-methylbutyl, 1,1-dimethylpropyl, and 1,2-dimethylpropyl.

As used herein, the term “(C₂-C₁₀)alkenyl” refers to straight chain and branched non-cyclic hydrocarbons having from 2 to 10 carbon atoms and including at least one carbon-carbon double bond. Representative straight chain and branched —(C₂-C₁₀)alkenyl groups include -vinyl, allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, and 3-hexenyl.

As used herein, the term “(C₂-C₅)alkenyl” refers to straight chain and branched non-cyclic hydrocarbons having from 2 to 5 carbon atoms and including at least one carbon-carbon double bond. Representative straight chain and branched —(C₂-C₅)alkyenyl groups include -vinyl, allyl, -1-butenyl, -2-butenyl, -isobutylenyl, -1-pentenyl, -2-pentenyl, -3-methyl-1-butenyl, and -2-methyl-2-butenyl.

As used herein, the term “(C₂-C₁₀)alkynyl” refers to straight chain and branched non-cyclic hydrocarbons having from 2 to 10 carbon atoms and including at least one carbon-carbon triple bond. Representative straight chain and branched C₂-C₁₀ alkynyl groups include -acetylenyl, -propynyl, -1 butynyl, -2-butynyl, -1-pentynyl, -2-pentynyl, -3-methyl-1-butynyl, -4-pentynyl, -1-hexynyl, -2-hexynyl, and -5-hexynyl.

As used herein, the term “—(C₂-C₅)alkynyl” refers to straight chain and branched non-cyclic hydrocarbons having from 2 to 5 carbon atoms and including at least one carbon-carbon triple bond. Representative straight chain and branched —(C₂-C₅)alkynyl groups include -acetylenyl, -propynyl, -1 butynyl, -2-butynyl, -1-pentynyl, -2-pentynyl, -3-methyl-1-butynyl, and -4-pentynyl.

As used herein, the term “(C₃-C₁₂)cycloalkyl” refers to cyclic saturated hydrocarbons having from 3 to 12 carbon atoms, and selected from cyclopropyl, cyclobutyl, cyclopentyl, cyclohexyl, cycloheptyl, cyclooctyl, and cyclononyl, cyclodecyl, cycloundecyl and cyclododecyl.

As used herein, the term “(C₃-C₁₂)cycloalkenyl” refers to cyclic hydrocarbons having from 3 to 12 carbon atoms, and including at least one carbon-carbon double bond, including cyclopropenyl, cyclobutenyl, cyclopentenyl, cyclohexenyl, cycloheptenyl, cyclooctenyl, and cyclononenyl, cyclodecenyl, cycloundecenyl and cyclododecenyl.

As used herein, the terms “halo” and “halogen” refer to fluoro, chloro, bromo or iodo.

“—(C₁-C₁₀)alkoxy” means straight chain and branched non-cyclic hydrocarbons having one or more ether groups and from 1 to 10 carbon atoms. Representative straight chain and branched (C₁-C₁₀)alkoxys include -methoxy, -ethoxy, propoxy, butyloxy, pentyloxy, hexyloxy, heptyloxy, methoxymethyl, 2-methoxyethyl, -5-methoxypentyl, 3-ethoxybutyl and the like.

“—(C₁-C₅)alkoxy” means straight chain and branched non-cyclic hydrocarbons having one or more ether groups and from 1 to 5 carbon atoms. Representative straight chain and branched (C₁-C₅)alkoxys include -methoxy, -ethoxy, propoxy, butyloxy, pentyloxy, methoxymethyl, 2-methoxyethyl, -5-methoxypentyl, 3-ethoxybutyl and the like.

“—CH₂(halo)” means a methyl group where one of the hydrogens of the methyl group has been replaced with a halogen. Representative —CH₂(halo) groups include —CH₂F, —CH₂Cl, —CH₂Br, and —CH₂I.

“—CH(halo)₂” means a methyl group where two of the hydrogens of the methyl group have been replaced with independently selected halogens. Representative —CH(halo)₂ groups include —CHF₂, —CHCl₂, —CHBr₂, —CHBrCl, —CHClI, and —CHI₂.

“—C(halo)₃” means a methyl group where each of the hydrogens of the methyl group has been replaced with independently selected halogens. Representative —C(halo)₃ groups include —CF₃, —CCl₃, —CBr₃, and —CI₃.

As used herein, the term “optionally substituted” refers to a group that is either unsubstituted or substituted.

Compounds useful in practicing the invention can be in the form of prodrugs of the compounds. Prodrugs are covalently bonded carrier molecules that release an active compound in vivo. Non-limiting examples of prodrugs typically include esters of the compounds of Formula I that can be metabolized to the active compound by the action of enzymes in the body. Such prodrugs may be prepared by reacting a compound of Formula I with an anhydride such as succinic anhydride.

Compounds useful in practicing the invention may contain one or more asymmetric centers, thus giving rise to enantiomers, diastereomers, and other stereoisomeric forms. The present invention encompasses the use of all such possible forms, as well as their racemic and resolved forms and mixtures thereof, and the uses thereof. The individual enantiomers may be separated according to methods known to those of ordinary skill in the art in view of the present disclosure. When such compounds described herein contain olefinic double bonds or other centers of geometric asymmetry, and unless specified otherwise, they include both E and Z geometric isomers. All tautomers are intended to be encompassed by the present invention as well.

The terms “a” and “an” refer to one or more.

Suitable anions (X⁻) for the Compounds according to Formula I include inorganic and organic anions such as, but are not limited to, sulfate; citrate; acetate; dichloroacetate; trifluoroacetate; oxalate; halide, such as chloride, bromide, iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; gluconate; glucoronate; saccharate; formate; mandelate; arginate; carboxylate; benzoate; glutamate; methanesulfonate; ethanesulfonate; benzenesulfonate; p-toluenesulfonate; and pamoate (i.e., 1,1′-methylene-bis-(2-hydroxy-3-naphthoate)). In case the charge of the anion is greater than required by the cation to yield a neutral compound, the anion is either present in an sub-stoichometric amount (e.g. only 0.5 SO₄²⁻ to neutralize a cation) to result a neutral compound or the remaining charge is neutralized by a further positive charged species such as H⁺, K⁺, Na⁺, Li⁺, etc (e.g. HSO₄²⁻— to neutralize a cation).

Compounds useful in practicing the invention also encompass solvates, such as the solvates of the compounds of Formula I. The term “solvate” as used herein is a combination, physical association and/or solvation of a compound with a solvent molecule such as, e.g. a disolvate, monosolvate or hemisolvate, where the ratio of solvent molecule to compound of Formula I is 2:1, 1:1 or 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. A compound of Formula I may be present as a solvated form with a pharmaceutically acceptable solvent, such as water, methanol, ethanol, and the like, and it is intended that the invention include both solvated and unsolvated forms of Formula I compound. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al., J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1): Article 12 (2004), and A. L. Bingham et al., Chem. Commun., 603-604 (2001). A typical, non-limiting, process of preparing a solvate would involve dissolving a compound of Formula I in a desired solvent (organic, water, or a mixture thereof) at temperatures above about 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

Preparation and Testing of Compounds

Compounds of Formula I and similar benzomorphan compounds can be made using conventional organic synthesis in view of the disclosure of US Patent Application Publication 2010/0324080. Opioid receptor binding assays and data, as well as [³⁵S]GTPγS functional receptor binding activities are described in US Patent Application Publication 2010/0324080.

As mentioned previously, three distinct opioid receptors have been identified: mu opioid receptors (also MOR); kappa opioid receptors (also KOR); and delta opioid receptors (also DOR). The location of these different receptors varies throughout the body. Mu receptors are probably the most studied: expression of mu receptors is 200 to 20,000 times higher in the brain than in certain epidermal cells. However, the compounds useful in practicing the invention may act either locally, peripherally or centrally. The affinities and effect of compounds useful for practicing the invention on each type of opioid receptor is characterized below. Conversion to a non-quaternary metabolite may aid the crossing of the blood-brain barrier and enhance the action on central receptors.

Typically, compounds useful in practicing the invention will have a high binding affinity for mu opioid receptors, i.e. a low MOR inhibitor constant, Ki (nM), of about 300 or less. In other embodiments, compounds useful in practicing the invention will have a MOR Ki (nM) of about 100 or less; about 10 or less; about 1 or less; or about 0.1 or less. Similarly, the compounds useful in practicing the invention typically will have a high binding affinity for kappa opioid receptors, i.e. a low KOR inhibitor constant, Ki (nM) of about 10,000 or less. In certain embodiments, compounds useful in practicing the invention will have a KOR Ki (nM) of about 5000 or less; about 1000 or less; about 500 or less; about 450 or less; about 350 or less; about 200 or less; about 100 or less; about 50 or less; or about 10 or less.

In contrast, compounds useful in practicing the invention will have a Ki (nM) for δ receptors of about 10 or more; or about 100 or more; or about 250 or more; or about 350 or more; or about 500 or more; or about 1000 or more; or about 2500 or more; or about 3000 or more; or about 4000 or more; or even about 10,000 or more.

Measures of a compound's activity at a given receptor are given by GTP Emax and GTP EC₅₀. GTP Emax (%) is the maximal effect elicited by a compound relative to the effect elicited by a standard, known agonist compound. GTP EC₅₀ is the concentration of a compound providing 50% of the maximal response for that compound at a given receptor. For MOR, the μ GTP Emax (%) is the maximal effect elicited by a compound relative to the effect elicited by the standard MOR agonist [D-Ala², N-methyl-Phe⁴ Gly-ol⁵]-enkephalin (a/k/a DAMGO). Generally, the μ GTP Emax (%) value measures the efficacy of a compound to treat or prevent pain or diarrhea. Typically, as μ-opioid antagonists, compounds useful in practicing the invention will have a μ GTP Emax (%) of less than about 50%. In certain embodiments, compounds useful in practicing the invention will have a μ GTP Emax (%) of less than about 40%; less than about 30%; less than about 20%; or less than about 10%. Compounds useful in practicing the invention will typically have a μ GTP EC₅₀ (nM) of about 5000 or less to stimulate μ-opioid receptor function. In certain embodiments, compounds useful in practicing the invention will have a μ GTP EC₅₀ (nM) of about 2000 or less; or about 1000 or less; or about 100 or less; or about 10 or less; or about 1 or less; or about 0.1 or less.

Similarly for KOR, the κ GTP Emax (%) is the maximal effect elicited by a compound relative to the effect elicited by the known κ-agonist, U69,593. Typically, compounds useful in practicing the invention will have a κ GTP Emax (%) of greater than about 30%. In certain embodiments, compounds useful in practicing the invention will have a κ GTP Emax (%) of greater than about 40%; of greater than about 50%; of greater than about 75%; greater than about 90%; or greater than about 100%. Compounds useful in practicing the invention typically will have a κ GTP EC₅₀ (nM) of about 10,000 or less to stimulate κ-opioid receptor function. In certain embodiments, compounds useful in practicing the invention will have a κ GTP EC₅₀ (nM) of about 5000 or less; about 2000 or less; about 1500 or less; about 1000 or less; about 600 or less; about 100 or less; about 50 or less; about 25 or less; or about 10 or less.

In like manner for DOR, δ GTP Emax (%) is the maximal effect elicited by a compound relative to the effect elicited by the known δ agonist, met-enkephalin. Although DOR binding is not thought critical, typically compounds useful in practicing the invention will have a δ GTP Emax (%) of from less than about 1% to about 110%. In certain embodiments, compounds useful in practicing the invention will have a δ GTP Emax (%) of less than about 5%; or less than about 10%; or less than about 20%; or less than about 50%; or less than about 75%; or less than about 90%; or less than about 100%; or less than about 110%. Compounds useful in practicing the invention typically have a δ GTP EC₅₀ (nM) of about 10,000 or more for stimulation of δ opioid receptor function. In certain embodiments, compounds useful in practicing the invention will have a δ GTP EC₅₀ (nM) of about 1000 or more; or about 100 or more; or about 90 or more; or about 50 or more; or about 25 or more; or about 10 or more.

In particular embodiments, compounds useful in practicing the invention have a mu Ki (nM) of less than 1000; a mu GTP EC₅₀ (nM) of less than 1000; a mu GTP Emax (%) of less than 50; a kappa Ki (nM) of less than 1000; a kappa GTP EC₅₀ (nM) of less than 1000; and a kappa GTP Emax (%) of greater than 50.

In other embodiments, certain compounds useful in practicing the invention have a mu Ki (nM) of less than 500; a mu GTP EC₅₀ (nM) of less than 500; a mu GTP Emax (%) of less than 20; a kappa Ki (nM) of less than 1000; a kappa GTP EC₅₀ (nM) of less than 500; and a kappa GTP Emax (%) of greater than 80%.

In other embodiments, certain compounds useful in practicing the invention have a mu Ki (nM) of less than 100; a mu GTP EC₅₀ (nM) of less than 100; a mu GTP Emax (%) of less than 10%; a kappa Ki (nM) of less than 100; a kappa GTP EC₅₀ (nM) of less than 100; and a kappa GTP Emax (%) of greater than 95%.

The receptor binding properties and functional properties of some specific compounds are illustrated below in the Examples.

Compositions and Combinations

Although compounds useful in practicing the invention can be administered to a mammal in the form of a raw chemical without any other components present, the compound is preferably administered as part of a pharmaceutical composition containing one or more antipruritic compounds in therapeutically effective amounts combined with a suitable pharmaceutically acceptable carrier. Such compositions are “compositions useful in practicing the invention” and they contain one or more antipruritic compounds that exhibit the properties of “compounds useful in practicing the invention.” The pharmaceutically acceptable carrier can be selected from pharmaceutically acceptable excipients and auxiliaries based on the route of administration. Pharmaceutical excipients are well known in the art, and examples of such excipients are described in US Patent Application Publication 2010/0324080. Thus, one aspect of the present invention includes pharmaceutical compositions comprising an effective amount of one or more compounds useful in practicing the invention, formulated with one or more pharmaceutically acceptable excipients.

As used herein, “a therapeutically effective amount” of a compound or composition useful in practicing the invention refers to that amount of the compound or composition effective for treating, ameliorating or preventing pruritus, by (a) detectably inhibiting or antagonizing mu opioid receptor function in a cell; (b) detectably activating or agonizing kappa opioid receptor function in a cell; or (c) both inhibiting mu opioid receptor function and activating kappa opioid receptor function in a cell.

In one embodiment, the compound is present in a composition in a therapeutically effective amount to achieve its intended therapeutic purpose. While individual needs may vary, a determination of optimal ranges of effective amounts of each compound is within the skill of the art. Typically, a compound useful in practicing the invention is administered to a mammal, e.g. a human, orally at a dose of from about 0.0025 to about 1500 mg per kg body weight of the mammal, or an equivalent amount of a pharmaceutically acceptable salt, prodrug or solvate thereof, per day to alleviate pruritus. A useful oral dose of a compound of the present invention administered to a mammal is from about 0.025 to about 50 mg per kg body weight of the mammal, or an equivalent amount of the pharmaceutically acceptable salt, prodrug or solvate thereof. A unit oral dose may comprise from about 0.01 to about 50 mg, and preferably from about 0.1 to about 10 mg, of a compound. The unit dose can be administered one or more times daily, e.g. as one or more tablets or capsules, each containing from about 0.01 mg to about 50 mg of the compound, or an equivalent amount of a pharmaceutically acceptable salt, prodrug or solvate thereof. The unit dose can be administered once a day, or once every 12 hours, or once every 8 hours, or once every six hours, or once every 4 hours, or as needed.

The methods of the present invention are primarily directed to treatment of human subjects suffering from, or at risk of suffering from, a pruritic condition. However, the methods of the present invention can be administered to any animal that may experience the beneficial effects of the present invention. Foremost among such animals are mammals, e.g., humans and companion animals.

The methods of the present invention can be carried out by administration of a compound useful in practicing the invention, or pharmaceutical composition useful in practicing the invention, via any effective route of administration. The choice of route of administration will vary depending upon the circumstances of the particular subject, and taking into account such factors as age, gender, health, and weight of the recipient, condition or disorder to be treated, type of concurrent treatment (if any), the frequency of treatment, and the nature and extent of the desired effect.

In one embodiment, a pharmaceutical composition useful in practicing the invention can be administered orally and is formulated into tablets, dragees, capsules or an oral liquid preparation. In one embodiment, the oral formulation comprises extruded multiparticulates comprising the compound of the invention. In another embodiment, a pharmaceutical composition useful in practicing the present invention is formulated to be administered rectally, i.e., as suppositories. In another embodiment, a pharmaceutical composition of the present invention is formulated to be administered by injection, such as intraveneously, intramuscularly, subcutaneously or intrathecally.

In another embodiment, a pharmaceutical composition useful in practicing the invention is formulated to be administered topically, for example as a cream, lotion, ointment, gel, spray, solution or patch. The topical pharmaceutical compositions may be formulated as an aqueous solution, suspension, lotion, gel, cream ointment, adhesive film and the like, with pharmaceutically acceptable excipients such as aloe vera, propylene glycol, DMSO, lecithine base, and the like. A gel excipient may comprise one or more of the following-petrolatum, lanoline, polyethylene glycols, bee wax, mineral oil, diluents, such as water and alcohol, and emulsifiers and stabilizers.

Pharmaceutical excipients for a pharmaceutical composition may vary, the choice of excipients being guided by the intended route of administration, but excipients are well known to those skilled in the art, see e.g. Remington, The Science and Practice of Pharmacy, 21^{st} Ed., 2005, University of the Sciences in Philadelphia, Publ. Lippincott Williams & Wilkins, incorporated by reference. For example, injectable formulations must generally be sterile; oral formulations may be protected from acidity in the stomach; and topical formulations may be placed in cream or ointment bases that facilitate transport of the drug into the skin.

Aqueous suspensions can contain the compounds in admixture with pharmaceutically acceptable excipients such as suspending agents, e.g., sodium carboxymethyl cellulose, methylcellulose, hydroxypropylmethylcellulose, sodium alginate, polyvinylpyrrolidone, gum tragacanth and gum acacia; dispersing or wetting agents such as naturally occurring phosphatide, e.g., lecithin, or condensation products of an alkylene oxide with fatty acids, e.g., polyoxyethylene stearate, or condensation products of ethylene oxide with long, chain aliphatic alcohols, e.g., heptadecaethyleneoxycetanol, or condensation products ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylenes sorbitan monooleate. Such aqueous suspensions can also contain one or more preservatives, e.g., ethyl or n-propyl-p-hydroxy benzoate.

Dispersible powders and granules suitable for preparation of an aqueous suspension by the addition of water provide the compounds in admixture with a dispersing or wetting agent, suspending agent and one or more preservatives, as is known in the art of drug formulation.

A topical formulation delivers a therapeutic effect at local and/or pheripheral opioid receptors and is not necessarily expected or required to deliver the active ingredients systemically to the bloodstream or to the central nervous system (brain and spinal cord) opioid receptors in the treated mammals. Topical administration of the pharmaceutical composition may be accomplished by application of a solution, gel, lotion, ointment, cream or other vehicle topically used to deliver therapeutics to a local site. One means of application is by spraying the composition over the area to be treated. In another embodiment, a patch which provides a sustained release topical formulation may also be used to administer the topical therapeutic. The patch may be a reservoir and porous membrane type or a solid matrix as are known in the art. The active agents may be in a plurality of microcapsules distributed throughout the permeable adhesive layer.

Compositions useful for practicing this invention can be provided in delayed, prolonged or sustained-release dosage formulations, or in immediate release formulations, as are known in the art.

A pharmaceutical composition useful for practicing the invention can contain from about 0.01 to 99 percent by weight, and preferably from about 0.25 to 75 percent by weight, of active compound(s).

In some embodiments, a compound useful in practicing the invention may be combined with other pharmaceutically active ingredients for combination therapy. For example, a compound useful in practicing the invention (i.e., the first therapeutic agent) and a second therapeutic agent can act additively or synergistically to treat the same condition. Alternatively, the first and second therapeutic agents can be used to treat different conditions, and may show no additive or synergistic action. In one embodiment, a compound useful in practicing the invention may be used as a first therapeutic agent to offset the side effects of a second therapeutic agent; as, for example, when a compound useful in practicing the invention is administered to relieve pruritus associated with opioid analgesic therapy.

In one embodiment, a compound useful in practicing the invention is administered to the patient concurrently with the second therapeutic agent; for example, in a single composition comprising an effective amount of a compound useful in practicing the invention and a second therapeutic agent. Accordingly, the present invention further provides a pharmaceutical composition comprising a combination of an effective amount of a compound useful in practicing the invention, an effective amount of a second therapeutic agent, and a pharmaceutically acceptable carrier. Alternatively, a compound useful in practicing the invention and the second therapeutic agent can be concurrently administered in separate compositions. In another embodiment, a compound useful in practicing the invention is administered prior or subsequent to administration of the second therapeutic agent. In this embodiment, the compound useful in practicing the invention is administered while the second therapeutic agent exerts its therapeutic effect, or the second therapeutic agent is administered while the compound useful in practicing the invention exerts its therapeutic effect.

In a particular embodiment, the second therapeutic agent is a mu opioid agonist, since a primary benefit of the present invention is to alleviate pruritus otherwise caused by mu agonist analgesic therapy. Examples of useful mu opioid agonists include, but are not limited to, alfentanil, allylprodine, alphaprodine, benzylmorphine, buprenorphine, codeine, desomorphine, dextromoramide, diamorphone, dihydrocodeine, dihydromorphine, ethylmorphine, etorphin, fentanyl, heroin, hydrocodone, hydromorphone, isomethadone, ketobemidone, levorphanol, lofentanil, meperidine, methadone, morphine, nicomorphine, normethadone, normorphine, opium, oxycodone, oxymorphone, propoxyphene, sufentanil, tilidine, tramadol, pharmaceutically acceptable salts thereof, and mixtures thereof. In certain embodiments, the opioid agonist is selected from buprenorphine, codeine, hydromorphone, hydrocodone, oxycodone, dihydrocodeine, dihydromorphine, morphine, tramadol, oxymorphone, pharmaceutically acceptable salts thereof, and mixtures thereof.

Alternatively, the second therapeutic agent can be a non-opioid analgesic such as, e.g., a non-steroidal anti-inflammatory agent (NSAID), an anti-migraine agent, an anti-emetic agent, a Cox-II inhibitor, a lipoxygenase inhibitor, a β-adrenergic blocker, an anti-convulsant, an anti-depressant, an anti-cancer agent, an agent for treating addictive disorder, an agent for treating Parkinson's disease and parkinsonism, an agent for treating anxiety, an agent for treating epilepsy, an agent for treating a seizure, an agent for treating stroke, an agent for treating constipation, an agent for treating psychosis, an agent for treating ALS, an agent for treating a cognitive disorder, an agent for treating dyskinesia, a mu agonist agent, or a mixture thereof. Useful second therapeutic agents in these categories are known to those skilled in the art, and mentioned in US Patent Application Publication 2010/0324080, and references cited therein, all incorporated by reference.

Effective amounts of the second therapeutic agents will generally be ascertainable by those skilled in the art depending on the identity of the second therapeutic agent and the severity of the condition being treated.

In a variation, a pharmaceutical composition or formulation may contain more than one compound useful for practicing the invention. In this variation, the activity profile of the first and second compound need not be identical. For example, the binding affinities of a first compound may differ in degree from a second compound relative to the mu, kappa and delta opioid receptors. Additionally, in this variation the first and second compounds may differ in the degree of KOR agonism or the in the degree of MOR antagonism. In a particular embodiment, a single compound useful for practicing the invention possesses the dual activity of KOR agonism and MOR antagonism.

The invention has been described above and in appended claims. Without intending to limit the scope of the invention, some specific, illustrative examples are described below.

### EXAMPLES

### Example 1

### Binding Assay Procedures

Binding assays are performed as follows and results are provided below in Table 1, below.

μ-opioid Receptor Binding Assay Procedures:

Radioligand dose-displacement binding assays for μ-opioid receptors used 0.2 nM[³H]-diprenorphine (NEN, Boston, Mass.), with 5-20 mg membrane protein/well in a final volume of 500 μl binding buffer (10 mM MgCl₂, 1 mM EDTA, 5% DMSO, 50 mM HEPES, pH 7.4). Reactions were carried out in the absence or presence of increasing concentrations of unlabeled naloxone. All reactions were conducted in 96-deep well polypropylene plates for 1-2 hr at room temperature. Binding reactions were terminated by rapid filtration onto 96-well Unifilter GF/C filter plates (Packard, Meriden, Conn.) presoaked in 0.5% polyethylenimine using a 96-well tissue harvester (Brandel, Gaithersburg, Md.) followed by performing three filtration washes with 500 μl of ice-cold binding buffer. Filter plates were subsequently dried at 50° C. for 2-3 hours. BetaScint scintillation cocktail (Wallac, Turku, Finland) was added (50 μl/well), and plates were counted using a Packard Top-Count for 1 min/well. The data were analyzed using the one-site competition curve fitting functions in GraphPad PRISM v. 3.0 (San Diego, Calif.), or an in-house function for one-site competition curve-fitting.

κ-opioid Receptor Binding Assay Procedures:

Membranes from recombinant HEK-293 cells expressing the human kappa opioid receptor (kappa) (cloned in house) were prepared by lysing cells in ice cold hypotonic buffer (2.5 mM MgCl₂, 50 mM HEPES, pH 7.4) (10 mL/10 cm dish) followed by homogenization with a tissue grinder/Teflon pestle. Membranes were collected by centrifugation at 30,000×g for 15 min at 4° C. and pellets were resuspended in hypotonic buffer to a final concentration of 1-3 mg/mL. Protein concentrations were determined using the BioRad protein assay reagent with bovine serum albumen as standard. Aliquots of kappa receptor membranes were stored at −80° C.

Radioligand dose displacement assays used 0.4-0.8 nM [³H]-U69,593 (NEN; 40 Ci/mmole) with 10-20 μg membrane protein (recombinant kappa opioid receptor expressed in HEK 293 cells; in-house prep) in a final volume of 200 μl binding buffer (5% DMSO, 50 mM Trizma base, pH 7.4). Non-specific binding was determined in the presence of 10 μM unlabeled naloxone or U69,593. All reactions were performed in 96-well polypropylene plates for 1 hr at a temperature of about 25° C. Binding reactions were determined by rapid filtration onto 96-well Unifilter GF/C filter plates (Packard) presoaked in 0.5% polyethylenimine (Sigma). Harvesting was performed using a 96-well tissue harvester (Packard) followed by five filtration washes with 200 μl ice-cold binding buffer. Filter plates were subsequently dried at 50° C. for 1-2 hours. Fifty μl/well scintillation cocktail (MicroScint20, Packard) was added and plates were counted in a Packard Top-Count for 1 min/well.

δ-opioid Receptor Binding Assay Procedures:

δ-opioid Receptor Binding Assay Procedures can be conducted as follows. Radioligand dose-displacement assays use 0.2 nM [³H]-Naltrindole (NEN; 33.0 Ci/mmole) with 10-20 μg membrane protein (recombinant delta opioid receptor expressed in CHO—K1 cells; Perkin Elmer) in a final volume of 500 μl binding buffer (5 mM MgCl₂, 5% DMSO, 50 mM Trizma base, pH 7.4). Non-specific binding is determined in the presence of 25 μm M unlabeled naloxone. All reactions are performed in 96-deep well polypropylene plates for 1 hr at a temperature of about 25° C. Binding reactions are determined by rapid filtration onto 96-well Unifilter GF/C filter plates (Packard) presoaked in 0.5% polyethylenimine (Sigma). Harvesting is performed using a 96-well tissue harvester (Packard) followed by five filtration washes with 500 μl ice-cold binding buffer. Filter plates are subsequently dried at 50° C. for 1-2 hours. Fifty μl/well scintillation cocktail (MicroScint20, Packard) is added and plates are counted in a Packard Top-Count for 1 min/well.

TABLE 1
Binding Efficacy of Benzomorphan Compounds

| Ref. | | Ki [mean ± SEM] (nM) | | |
|---|---|---|---|---|
| No. | Compound | μ | δ | κ |
| 1 | 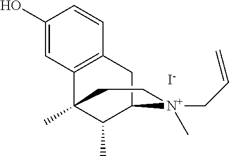 | 56.45 ± 9.11 | | 10.3 ± 3.03 |

### Example 2

### Functional Assay Procedures

Functional assays are performed as follows and results are provided below in Table 2, below.

μ-Opioid Receptor Functional Assay Procedures:

[³⁵S]GTPγS functional assays were conducted using freshly thawed μ-receptor membranes. Assay reactions were prepared by sequentially adding the following reagents to binding buffer (100 mM NaCl, 10 mM MgCl₂, 20 mM HEPES, pH 7.4) on ice (final concentrations indicated): membrane protein (0.026 mg/mL), saponin (10 mg/mL), GDP (3 mM) and [³⁵S]GTPγS (0.20 nM; NEN). The prepared membrane solution (190 μl/well) was transferred to 96-shallow well polypropylene plates containing 10 μl of 20× concentrated stock solutions of the agonist DAMGO prepared in dimethyl sulfoxide (DMSO). Plates were incubated for 30 min at about 25° C. with shaking. Reactions were terminated by rapid filtration onto 96-well Unifilter GF/B filter plates (Packard, Meriden, Conn.) using a 96-well tissue harvester (Brandel, Gaithersburg, Md.), followed by three filtration washes with 200 μl of ice-cold wash buffer (10 mM NaH₂PO₄, 10 mM Na₂HPO₄, pH 7.4). Filter plates were subsequently dried at 50° C. for 2-3 hr. BetaScint scintillation cocktail (Wallac, Turku, Finland) was added (50 μl/well) and plates were counted using a Packard Top-Count for 1 min/well. Data were analyzed using the sigmoidal dose-response curve fitting functions in GraphPad PRISM v. 3.0, or an in-house function for non-linear, sigmoidal dose-response curve-fitting.

κ-Opioid Receptor Functional Assay Procedures:

Functional [³⁵S]GTPγS binding assays were conducted as follows. Kappa opioid receptor membrane solution was prepared by sequentially adding final concentrations of 0.026 μg/μl kappa membrane protein (in-house), 10 μg/mL saponin, 3 μM GDP and 0.20 nM [³⁵S]GTPγS to binding buffer (100 mM NaCl, 10 mM MgCl₂, 20 mM HEPES, pH 7.4) on ice. The prepared membrane solution (190 μl/well) was transferred to 96-shallow well polypropylene plates containing 10 μl of 20× concentrated stock solutions of agonist prepared in DMSO. Plates were incubated for 30 min at a temperature of about 25° C. with shaking. Reactions were terminated by rapid filtration onto 96-well Unifilter GF/B filter plates (Packard) using a 96-well tissue harvester (Packard) and followed by three filtration washes with 200 μl ice-cold binding buffer (10 mM NaH₂PO₄, 10 mM Na₂HPO₄, pH 7.4). Filter plates were subsequently dried at 50° C. for 2-3 hours. Fifty μl/well scintillation cocktail (MicroScint20, Packard) was added and plates were counted in a Packard Top-Count for 1 min/well.

δ-Opioid Receptor Functional Assay Procedures:

Functional [³⁵S]GTPγS binding assays can be conducted as follows. Delta opioid receptor membrane solution is prepared by sequentially adding final concentrations of 0.026 μg/μl delta membrane protein (Perkin Elmer), 10 μg/mL saponin, 3 μM GDP and 0.20 nM [³⁵S]GTPγS to binding buffer (100 mM NaCl, 10 mM MgCl₂, 20 mM HEPES, pH 7.4) on ice. The prepared membrane solution (190 μl/well) is transferred to 96-shallow well polypropylene plates containing 10 μl of 20× concentrated stock solutions of agonist prepared in DMSO. Plates are incubated for 30 min at a temperature of about 25° C. with shaking. Reactions are terminated by rapid filtration onto 96-well Unifilter GF/B filter plates (Packard) using a 96-well tissue harvester (Packard) and followed by three filtration washes with 200 μl ice-cold binding buffer (10 mM NaH₂PO₄, 10 mM Na₂HPO₄, pH 7.4). Filter plates are subsequently dried at 50° C. for 1-2 hours. Fifty μl/well scintillation cocktail (MicroScint20, Packard) is added and plates are counted in a Packard Top-count for 1 min/well.

TABLE 2
Activity Response of Benzomorphan Compounds

| | | GTPγS (EC₅₀: nM, Eₘₐₓ: %) [mean ± SEM] | | | | | |
|---|---|---|---|---|---|---|---|
| Ref. | | μ | | δ | | κ | |
| No. | Compound | EC₅₀ | Eₘₐₓ | EC₅₀ | Eₘₐₓ | EC₅₀ | Eₘₐₓ |
| 1 | 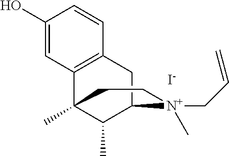 | >20 μM | 3.67 ± 1.33 | | | 268.26 ± 46.44 | 27.33 ± 3.38 |

### Example 3

### In Vivo Itch Response Assay

In Vivo Model and Procedures:

An in vivo model was devised and implemented to evaluate the antipruritic activity of compounds in mice. Compound 48/80 (Sigma Chemical), a known pruritogen in mice, was mixed in a 0.9% saline vehicle and administered to various cohorts of adult male CD-1 mice by s.c. injection at the nape of the neck at dosage levels of 12.5, 25, 50 and 100 μg. The saline vehicle alone was used as a control. The mice were monitored visually and scratching bouts were counted over the ensuing 30 minutes. As expected, the control produced the fewest scratching bouts, while successively higher doses of the pruritogen 48/80 produced increasing numbers of scratching bouts on average.

The model was verified by establishing that a known kappa agonist, antipruritic compound, Nalfurafine HCl (REMITCH®, Purdue Pharma) caused a dose dependent reduction in scratch response, when given by s.c. injection in the rear flank 20 minutes prior to administration of a 50 μg dose of pruritogenic compound 48/80 in the nape as described above. The Nalfurafine was shown to reduce the scratching bout responses in a dose-dependent manner.

Experimental Compounds:

Next, test benzomorphan Compound #1 (see Examples 1 and 2 above) was given in various doses (Table 3 below) in the rear flank 20 minutes prior to pruritogen injection in the same manner as the Nalfurafine. Scratching bouts were again counted for the ensuing 30 minute period, and the data are presented in Table 3.

TABLE 3
Scratch Response with Benzomorphan Compounds

| | Total # Scratching |
|---|---|
| | Bouts in 30 mins |
| Treatment Groups | (mean ± S.E.M.) |
| Vehicle + Vehicle | 26.1 ± 4.20 |
| Vehicle + Compound 48/80 (0.5 mg/kg) | 170.3 ± 18.66^{a} |
| 3 mg/kg Compound #1 + Compound 48/80 | 155.8 ± 13.73^{a} |
| (0.5 mg/kg) | |
| 10 mg/kg Compound #1 + Compound 48/80 | 129.4 ± 16.18^{b} |
| (0.5 mg/kg) | |
| 30 mg/kg Compound #1 + Compound 48/80 | 70.8 ± 19.74^{c} |
| (0.5 mg/kg) | |
| 0.04 mg/kg Nalfurafine + Compound 48/80 | 25.6 ± 7.92^{d} |
| (0.5 mg/kg) | |

^{a}statistically different from Vehicle + Vehicle group at P < 0.0001 significance,
^{b}statistically different from Vehicle + Vehicle group at P < 0.001 significance;
^{c}statistically different from Vehicle + Compound 48/80 group at P < 0.001 significance; and
^{d}statistically different from Vehicle + Compound 48/80 group at P < 0.0001 significance;

From the data above, it can be seen first that Compound 48/80 induced significant scratch response in the mice at P<0.0001 significance compared to the Vehicle+Vehicle group, thus confirming the validity of Compound 48/80 as a pruritogen in the model. Second, it can be seen that Compound #1 alleviated pruritus by reducing the mean number of scratching bouts initiated by pruritogen administration. The reduction in scratching response was statistically significant versus the Vehicle+Compound 48/80 group in two comparisons: (1) with Nalfurafine+Compound 48/80 group and (2) with the Test Compound #1+Compound 48/80 group at the 30 mg/kg dose. However, at a dose of 30 mg/kg, the test compound exhibits less potency than Nalfurafine at 0.04 mg/kg. Thus compounds useful in accordance with the invention have industrial application and utility.

Having now fully described this invention, it will be understood by those of ordinary skill in the art that the same can be performed within a wide and equivalent range of conditions, formulations and other parameters without affecting the scope of the invention or any embodiment thereof. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference in their entirety.

What is claimed is:

1. A method of treating, ameliorating or preventing pruritus in a patient in need thereof, comprising administering to the patient a pharmaceutical composition containing one or more antipruritic benzomorphan compounds in a therapeutically effective amount to cause both mu opioid receptor antagonism and kappa opioid receptor agonism
said benzomorphan compound having the structure of formula I, 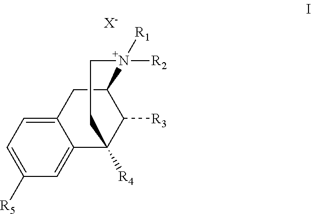
wherein R¹ and R² are each independently selected from the group consisting of —(C₁-C₁₀)alkyl, —(C₂-C₁₀)alkenyl, —(C₂-C₁₀)alkynyl, —(C₃-C₁₂)cycloalkyl, —(C₃-C₁₂)cycloalkenyl, —(CH₂)ₙ—O—(CH₂)ₙ—CH₃, (C₁-C₁₀)alkoxy, C(halo)₃, CH(halo)₂, CH₂(halo), C(O)R⁶, —C(O)O—(C₁-C₁₀)alkyl, and —(CH₂)ₙ—N(R⁷)₂, each of which is optionally substituted by 1, 2, or 3 independently selected R⁸ groups;
R³ and R⁴ are each independently selected from (a)—H; or (b)—(C₁-C₅)alkyl, —(C₂-C₅)alkenyl, and —(C₂-C₅)alkynyl;
R⁵ is selected from (a) —H, —OH, halo, —C(halo)₃, —CH(halo)₂, and—CH₂(halo) (b) —(C₁-C₅)alkyl, —(C₂-C₅)alkenyl, —(C₂-C₅)alkynyl, —(CH₂)ₙ—O—(CH₂)ₙ—CH₃, —(C₁-C₅)alkoxy, each of which is optionally substituted with 1, 2, or 3 independently selected R⁸ groups;
R⁶ is selected from —H, —(C₁-C₁₀)alkyl, —(C₂-C₁₀)alkenyl, —(C₂-C₁₀)alkynyl, and —(C₁-C₁₀)alkoxy;
each R⁷ is independently selected from —H, —(C₁-C₁₀)alkyl, —(C₂-C₁₀)alkenyl, and —(C₂-C₁₀)alkynyl;
each R⁸ is independently selected from —OH, halo, —(C₁-C₁₀)alkyl, —(C₂-C₁₀)alkenyl, —(C₂-C₁₀)alkynyl, —(C₁-C₁₀)alkoxy, —(C₃-C₁₂)cycloalkyl, —CHO, —C(O)OH, —C(halo)₃, —CH(halo)₂, CH₂(halo), and —(CH₂)ₙ—O—(CH₂)ₙ—CH₃;
X⁻ is a pharmaceutically acceptable organic or inorganic anion;
each n is independently selected from an integer from 0, 1, 2, 3, 4, 5, or 6; or a solvate or prodrug thereof,
provided that the compound is not

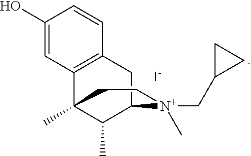

2. The method of claim 1, wherein the pruritus is associated with the administration of an opioid.

3. The method of claim 2, wherein the antipruritic benzomorphan compound is administered concurrently with an opioid.

4. The method of claim 1, wherein at least one of R¹ and R² is —(C₂-C₁₀)alkenyl.

5. The method of claim 1, wherein at least one of R¹ and R² is —(C₂-C₅)alkenyl.

6. The method of claim 4, wherein the antipruritic compound is:
3-allyl-9-hydroxy-3,6,11-trimethyl-1,2,3,4,5,6-hexahydro-2,6-methano-benzo[d]azocinium]; or a pharmaceutically acceptable salts, solvates or prodrug thereof.

7. The method of claim 3 wherein the opioid and the antipruritic compound are administered in a single composition.

8. The method of claim 7 wherein the opioid is selected from buprenorphine, codeine, hydromorphone, hydrocodone, oxycodone, dihydrocodeine, dihydromorphine, morphine, tramadol, oxymorphone, pharmaceutically acceptable salts thereof, and mixtures thereof.

9. The method of claim 1, wherein the antipruritic compound is administered by a topical route.

10. The method of claim 1, wherein the antipruritic compound is administered by an oral route.

11. The method of claim 1, wherein the antipruritic compound is administered for a pruritic condition that is not induced by opioid analgesic therapy.

12. The method according to claim 1, comprising administering a pharmaceutical composition exhibiting a mu opioid receptor GTP Emax of not more than about 30% and a kappa opioid receptor GTP Emax more than about 40%.

13. The method according to claim 12, comprising administering a pharmaceutical composition exhibiting a mu opioid receptor GTP Emax of not more than about 20% and a kappa opioid receptor GTP Emax more than about 75%.

14. The method according to claim 12, comprising administering a pharmaceutical composition exhibiting a mu opioid receptor GTP Emax of not more than about 10% and a kappa opioid receptor GTP Emax more than about 90%.

15. The method according to claim 1, comprising administering a pharmaceutical composition exhibiting a mu opioid receptor inhibitor constant, Ki, of about 300 nM or less, and a kappa opioid receptor inhibitor constant, Ki, of about 10,000 nM or less.

16. The method according to claim 15, comprising administering a pharmaceutical composition exhibiting a mu opioid receptor inhibitor constant, Ki, of about 100 nM or less, and a kappa opioid receptor inhibitor constant, Ki, of about 1,000 nM or less.

17. The method according to claim 13, comprising administering a pharmaceutical composition exhibiting a mu opioid receptor inhibitor constant, Ki, of about 100 nM or less, and a kappa opioid receptor inhibitor constant, Ki, of about 1,000 nM or less.
